(19) United States
(12) Patent Application Publication
(10) Pub. No.: US 2026/0337181 A1
(43) Pub. Date: Oct. 29, 2026

(54) SOLID STATE FORMS OF DENIFANSTAT

(71) Applicant: ASSIA CHEMICAL INDUSTRIES LTD., Tel Aviv (IL)

(72) Inventors: Anantha Rajmohan Muthusamy, Sivakasi, Tamil Nadu (IN); Meenakshi Sundaram Somasundaram, Virudhunagar DT, Tamil Nadu (IN); Laxman Gande, Suryapet, Telangana (IN)

(21) Appl. No.: 19/114,882

(22) PCT Filed: Sep. 29, 2023
(86) PCT No.: PCT/IB2023/059781

§ 371 Date: Mar. 25, 2025

(30) Foreign Application Priority Data
Oct. 1, 2022 (IN) 202211056470
Oct. 21, 2022 (IN) 202211060418

(51) Int. Cl. C07D 403/10; A61K 31/454

(52) U.S. Cl. CPC C07D 403/10; A61K 31/454; C07B 2200/13

(57) ABSTRACT

The present disclosure encompasses solid state forms of Denifanstat, in embodiments crystalline polymorphs of Denifanstat, processes for preparation thereof, and pharmaceutical compositions thereof.

### FIELD OF THE DISCLOSURE

The present disclosure encompasses solid state forms of Denifanstat, preferably crystalline Denifanstat, in embodiments crystalline polymorphs of Denifanstat, processes for preparation thereof, and pharmaceutical compositions thereof.

### BACKGROUND OF THE DISCLOSURE

Denifanstat, 4-(1-(4-cyclobutyl-2-methyl-5-(5-methyl-1H-1,2,4-triazol-3-yl)benzoyl) piperidin-4-yl)benzonitrile, has the following chemical structure:

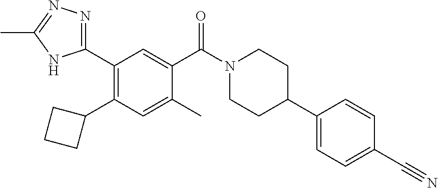 [SMILES: Cc1nnc(-c2cc(C(=O)N3CCC(c4ccc(C#N)cc4)CC3)c(C)cc2C2CCC2)[nH]1]

Denifanstat is an orally bioavailable fatty acid synthase (FASN) inhibitor, and it is developed for the treatment of Glioblastoma, Acne; Astrocytoma; Breast cancer, Non-alcoholic steatohepatitis, Non-small cell lung cancer, Liver disorders, Colorectal cancer, and Solid tumours.

The compound is described in U.S. Pat. No. 8,871,790.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Denifanstat.

### SUMMARY OF THE DISCLOSURE

The present disclosure provides crystalline polymorphs of Denifanstat, processes for preparation thereof, and pharmaceutical compositions thereof. These crystalline polymorphs can be used to prepare other solid state forms of Denifanstat, Denifanstat salts and their solid state forms.

The present disclosure also provides uses of the said solid state forms of Denifanstat in the preparation of other solid state forms of Denifanstat or salts thereof.

The present disclosure provides crystalline polymorphs of Denifanstat for use in medicine, including for the treatment of Glioblastoma, Acne; Astrocytoma; Breast cancer, Non-alcoholic steatohepatitis, Non-small cell lung cancer, Liver disorders, Colorectal cancer, and Solid tumours.

The present disclosure also encompasses the use of crystalline polymorphs of Denifanstat of the present disclosure for the preparation of pharmaceutical compositions and/or formulations.

In another aspect, the present disclosure provides pharmaceutical compositions comprising crystalline polymorphs of Denifanstat according to the present disclosure.

The present disclosure includes processes for preparing the above mentioned pharmaceutical compositions. The processes includes combining any one or a combination of the crystalline polymorphs of Denifanstat with at least one pharmaceutically acceptable excipient.

The crystalline polymorph of Denifanstat as defined herein and the pharmaceutical compositions or formulations of the crystalline polymorph of Denifanstat may be used as medicaments, such as for the treatment of Glioblastoma, Acne; Astrocytoma, Breast cancer; Non-alcoholic steatohepatitis, Non-small cell lung cancer, Liver disorders, Colorectal cancer; and Solid tumours.

The present disclosure also provides methods of treating Glioblastoma, Acne; Astrocytoma, Breast cancer; Non-alcoholic steatohepatitis, Non-small cell lung cancer, Liver disorders, Colorectal cancer, and Solid tumours by administering a therapeutically effective amount of any one or a combination of the crystalline polymorphs of Denifanstat of the present disclosure, or at least one of the above pharmaceutical compositions, to a subject suffering from Glioblastoma, Acne; Astrocytoma, Breast cancer; Non-alcoholic steatohepatitis, Non-small cell lung cancer, Liver disorders, Colorectal cancer and Solid tumours or otherwise in need of the treatment.

The present disclosure also provides uses of crystalline polymorphs of Denifanstat of the present disclosure, or at least one of the above pharmaceutical compositions, for the manufacture of medicaments for treating e.g. Glioblastoma, Acne; Astrocytoma, Breast cancer; Non-alcoholic steatohepatitis, Non-small cell lung cancer, Liver disorders, Colorectal cancer and Solid tumours.

### BRIEF DESCRIPTION OF THE DRAWINGS

FIG. 1 shows a characteristic X-ray powder diffraction pattern (XRPD) of Denifanstat Form DF1.

FIG. 2 shows a characteristic XRPD of a Denifanstat Form DF2.

FIG. 3 shows a characteristic XRPD of a Denifanstat Amorphous Form.

FIG. 4 shows a characteristic XRPD of a Denifanstat Form DF3.

FIG. 5a shows solid state ¹³C NMR spectrum of Form DF1 of Denifanstat (full scan).

FIG. 5b shows solid state ¹³C NMR spectrum of Form DF1 of Denifanstat (at the range of 0-100 ppm).

FIG. 5c shows solid state ¹³C NMR spectrum of Form DF1 of Denifanstat (at the range of 100-200 ppm).

### DETAILED DESCRIPTION OF THE DISCLOSURE

The present disclosure encompasses solid state forms of Denifanstat, particularly crystalline Denifanstat, including crystalline polymorphs of Denifanstat, processes for preparation thereof, and pharmaceutical compositions thereof. In embodiments, the present disclosure provides crystalline forms of Denifanstat designated as Form DF1, Form DF2 and DF3 (defined herein).

Solid state properties of Denifanstat and crystalline polymorphs thereof can be influenced by controlling the conditions under which Denifanstat and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Denifanstat described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Denifanstat. In some embodiments of the disclosure, the described crystalline polymorph of Denifanstat may contain from about 1% to about 20% (w/w), from about 5% to about 20% (w/w), or from about 5% to about 10% (w/w) of one or more other crystalline polymorph of the same Denifanstat. For example, a crystalline polymorph of Denifanstat according to any aspect or embodiment of the present invention may be polymorphically pure, and may contain: about 20% (w/w) or less, about 10% (w/w) or less, about 5% (w/w) or less, about 2% (w/w) or less, about 1% (w/w) or less, or about 0% of any other forms of Denifanstat, as measured, for example, by XRPD. Thus, for example, a crystalline polymorph of Denifanstat as described in any aspect or embodiment herein, which is polymorphically pure, may contain: about 20% (w/w) or less, about 10% (w/w) or less, about 5% (w/w) or less, about 2% (w/w) or less, about 1% (w/w) or less, or about 0% of any other forms of Denifanstat. As another example, a crystalline polymorph of Denifanstat as described in any aspect or embodiment herein, which is polymorphically pure, may contain: about 20% (w/w) or less, about 10% (w/w) or less, about 5% (w/w) or less, about 2% (w/w) or less, about 1% (w/w) or less, or about 0% of any other forms of the Denifanstat. Alternatively, a crystalline polymorph of Denifanstat according to any aspect or embodiment of the present invention may be polymorphically pure and may contain greater than about 80% (w/w), greater than about 90% (w/w), greater than about 95% (w/w), greater than about 98% (w/w), greater than about 99% (w/w), or about 100% of the crystalline polymorph of Denifanstat.

The solid state forms of Denifanstat as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w), about 0.8% (w/w) or less, about 0.6% (w/w) or less, about 0.4% (w/w) or less, about 0.2% (w/w) or less, about 0.1% (w/w) or less, or about 0% of any other compound as measured, for example, by HPLC. Alternatively, a chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent, or less about 0.2% area percent or less, about 0.1% area percent or less or about 0% of any other compound as measured by HPLC. Alternatively, a chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC. Thus, pure or purified Denifanstat described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2%, (w/w) greater than about 99.4% (w/w), greater than about 99.6% (w/w), greater than about 99.8% (w/w), greater than about 99.9% (w/w), or about 100% of the subject Denifanstat. Alternatively, pure or purified Denifanstat described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2%, area percent, greater than about 99.4% area percent, greater than about 99.6% area percent, greater than about 99.8% (area percent, greater than about 99.9% area percent, or about 100% of the subject Denifanstat. The chemical purity may be determined using any suitable process, for example, using HPLC and UV detection (e.g. at 220 nm).

Depending on which other crystalline polymorphs a comparison is made, the crystalline polymorphs of Denifanstat of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Denifanstat referred to herein as being characterized by graphical data “as depicted in” or “as substantially depicted in” a Figure will thus be understood to include any crystal forms of Denifanstat characterized with the graphical data having such small variations, as are well known to the skilled person, in comparison with the Figure.

As used herein, and unless stated otherwise, the term “anhydrous” in relation to crystalline forms of Denifanstat, relates to a crystalline form of Denifanstat which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Denifanstat of the present disclosure corresponds to a crystalline polymorph of Denifanstat that is physically separated from the reaction mixture in which it is formed.

As used herein, unless stated otherwise, the XRPD measurements are taken using copper Kα radiation wavelength 1.5418 Å. XRPD peaks reported herein are measured using Cuk a radiation, λ=1.5418 Å, typically at a temperature of 25±3° C.

As used herein, unless stated otherwise, solid state ¹³C NMR data is obtained using ¹³C CP/MAS NMR method. Particularly, as used herein, unless stated otherwise, the ¹³C CP/MAS NMR reported herein are measured at 500 MHz, preferably at a temperature of at 298 K±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The present disclosure includes a crystalline Denifanstat.

The crystalline Denifanstat may be characterized by an X-ray powder diffraction pattern having peaks at 19.8, 23.4 and 25.4 degrees 2-theta±0.2 degrees 2-theta.

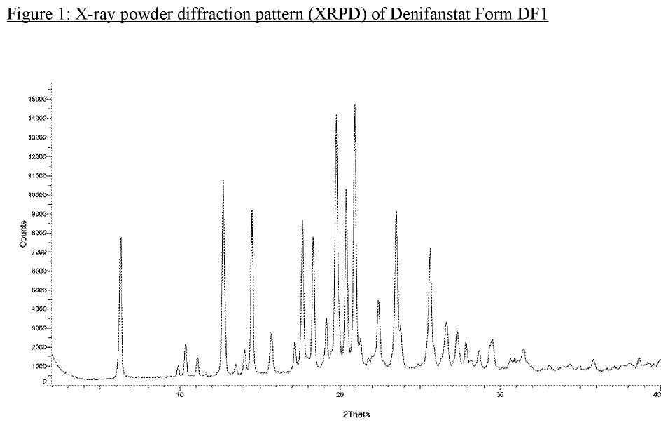

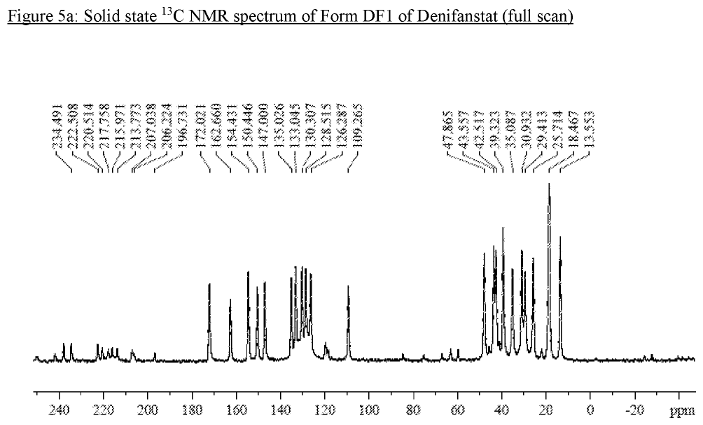

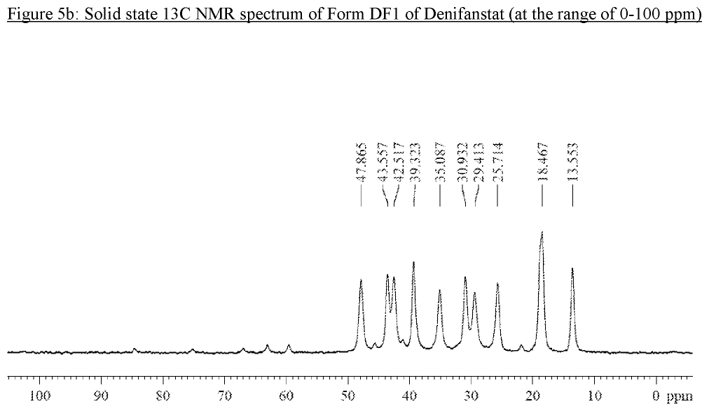

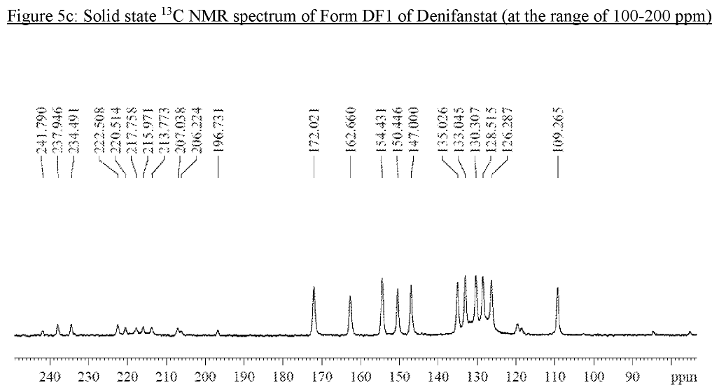

The present disclosure includes a crystalline polymorph of Denifanstat, designated DF1. The crystalline Form DF1 of Denifanstat may be characterized by data selected from one or more of the following: an X-ray powder diffraction pattern substantially as depicted in FIG. 1; an X-ray powder diffraction pattern having peaks at 19.8, 23.4 and 25.4 degrees 2-theta±0.2 degrees 2-theta; a solid state ¹³C NMR spectrum having peaks at 13.6, 25.7, 35.1, 39.3, 47.9, 109.3, 135.0, 147.0, 150.4, 154.4, 162.7 and 172.0 ppm±0.2 ppm; a solid state ¹³C NMR spectrum having the following chemical shift absolute differences from a reference peak at 18.5 ppm±2 ppm of 4.9, 7.2, 16.6, 20.8, 29.4, 90.8, 116.5, 128.5, 131.9, 135.9, 144.2 and 153.5 ppm±0.1 ppm; a solid state ¹³C NMR spectrum substantially as depicted in FIG. 5a, 5b or 5c; and combinations of these data.

Crystalline Form DF1 of Denifanstat may be further characterized by an X-ray powder diffraction pattern having peaks at 19.8, 23.4 and 25.4 degrees 2-theta±0.2 degrees 2-theta, and also having any one, two, three, four, or five additional peaks selected from 6.3, 14.5, 18.3, 20.4 and 20.9 degrees 2-theta±0.2 degrees 2-theta. Optionally, crystalline Form DF1 of Denifanstat may be characterized by an X-ray powder diffraction pattern having peaks at: 6.3, 14.5, 18.3, 19.8, 20.4, 20.9, 23.4, and 25.4 degrees 2-theta±0.2 degrees 2-theta.

Alternatively, crystalline Form DF1 of Denifanstat may be characterized by an X-ray powder diffraction pattern having peaks at 6.3, 14.5, 18.3, 20.4 and 20.9 degrees 2-theta±0.2 degrees 2-theta. Crystalline Form DF1 of Denifanstat may be further characterized by an X-ray powder diffraction pattern having peaks at 6.3, 14.5, 18.3, 20.4 and 20.9 degrees 2-theta±0.2 degrees 2-theta, and also having any one, two, three, or four additional peaks selected from 12.7, 17.7, 19.8 and 22.4 degrees 2-theta±0.2 degrees 2-theta.

Crystalline Form DF1 of Denifanstat may be characterized by an X-ray powder diffraction pattern having peaks at 6.3, 12.7, 14.5, 17.7, 18.3, 19.8, 20.4, 20.9 and 22.4 degrees 2-theta±0.2 degrees 2-theta.

Crystalline Form DF1 of Denifanstat may be characterized by an X-ray powder diffraction pattern having peaks at 6.3, 12.7, 14.5, 17.7, 18.3, 19.8, 20.4, 20.9, 22.4, 23.4 and 25.4 degrees 2-theta±0.2 degrees 2-theta.

In any aspect or embodiment of the present disclosure, crystalline Form DF1 of Denifanstat may be characterized by an X-ray powder diffraction pattern as described in any of the embodiments described herein, and wherein the X-ray powder diffraction pattern also has an absence of peaks at: 0 to 5.8 degrees 2-theta±0.2 degrees 2-theta, or 2.0 to 5.8 degrees 2-theta±0.2 degrees 2-theta, or 2.0 to 5.5 degrees±0.2 degrees 2-theta. Alternatively or additionally, according to any aspect or embodiment of the present disclosure, crystalline Form DF1 of Denifanstat may be characterized by an X-ray powder diffraction pattern as described in any of the embodiments described herein, and wherein the X-ray powder diffraction pattern also has an absence of peaks at: 6.8 to 9.4 degrees 2-theta±0.2 degrees 2-theta, or 7.0 to 9.0 degrees 2-theta±0.2 degrees 2-theta.

In any aspect of aspect or embodiment of the present disclosure, crystalline DF1 of Denifanstat may be characterized by:
an X-ray powder diffraction pattern having peaks at: 19.8, 23.4 and 25.4 degrees 2-theta±0.2 degrees 2-theta; or
an X-ray powder diffraction pattern having peaks at: 19.8, 23.4 and 25.4 degrees 2-theta±0.2 degrees 2-theta, and also having any one, two, three, four, or five additional peaks selected from 6.3, 14.5, 18.3, 20.4 and 20.9 degrees 2-theta #0.2 degrees 2-theta;
or an X-ray powder diffraction pattern having peaks at: 6.3, 14.5, 18.3, 19.8, 20.4, 20.9, 23.4, and 25.4 degrees 2-theta±0.2 degrees 2-theta;
or an X-ray powder diffraction pattern having peaks at: 6.3, 14.5, 18.3, 20.4 and 20.9 degrees 2-theta±0.2 degrees 2-theta; or an X-ray powder diffraction pattern having peaks at 6.3, 14.5, 18.3, 20.4 and 20.9 degrees 2-theta±0.2 degrees 2-theta, and also having any one, two, three, or four additional peaks selected from 12.7, 17.7, 19.8 and 22.4 degrees 2-theta±0.2 degrees 2-theta; or
an X-ray powder diffraction pattern having peaks at: 6.3, 12.7, 14.5, 17.7, 18.3, 19.8, 20.4, 20.9 and 22.4 degrees 2-theta±0.2 degrees 2-theta; or
an X-ray powder diffraction pattern having peaks at: 6.3, 12.7, 14.5, 17.7, 18.3, 19.8, 20.4, 20.9, 22.4, 23.4 and 25.4 degrees 2-theta±0.2 degrees 2-theta;
   wherein in each case, the X-ray powder diffraction pattern has an absence of peaks at 0 to 5.8 degrees 2-theta±0.2 degrees 2-theta (particularly at 2.0 to 5.5 degrees±0.2 degrees 2-theta) and/or at: 6.8 to 9.4 degrees 2-theta±0.2 degrees 2-theta (particularly 7.0 to 9.0 degrees 2-theta±0.2 degrees 2-theta).

In any aspect or embodiment of the present disclosure, crystalline Form DF1 of Denifanstat is preferably isolated. In any aspect or embodiment of the present disclosure, crystalline Form DF1 of Denifanstat is an anhydrous form.

In one embodiment of the present disclosure, crystalline Form DF1 of Denifanstat is isolated.

Crystalline Form DF1 of Denifanstat may be anhydrous form.

Crystalline Form DF1 of Denifanstat may be characterized by each of the above characteristics alone/or by all possible combinations, e.g., an XRPD pattern having peaks at 19.8, 23.4 and 25.4 degrees 2-theta±0.2 degrees 2-theta; an XRPD pattern having peaks at 6.3, 14.5, 18.3, 20.4 and 20.9 degrees 2-theta±0.2 degrees 2-theta; an XRPD pattern as depicted in FIG. 1, and combinations thereof.

Crystalline Form DF1 of Denifanstat according to any aspect or embodiment of the disclosure may be polymorphically pure or as substantially free of any other solid state (or polymorphic) forms of Denifanstat.

The present disclosure further comprises a process for preparation of Form DF1 of Denifanstat. The process may comprise slurrying amorphous Denifanstat in a solvent, preferably wherein the solvent is a C₄ to C₆ ether (preferably diethyl ether or diisopropyl ether, and more preferably diisopropyl ether), for a sufficient time to form Denifanstat Form DF1; and optionally isolating the Form DF1 of Denifanstat. Form DF1 of Denifanstat as described in any aspect or embodiment of the disclosure, may be prepared by a process comprising: combining Denifanstat in the solvent to form a mixture, and maintaining the mixture for a sufficient time for prepare Form DF1 of Denifanstat. Particularly, Form DF1 of Denifanstat as described in any aspect or embodiment of the disclosure, may be prepared by a process comprising:
(i) providing a suspension of amorphous Denifanstat in diisopropyl ether;
(ii) stirring the suspension;
(iii) optionally isolating Form DF1 of Denifanstat; and
(iv) optionally drying the Form DF1 of Denifanstat.

In any embodiment of this process, the suspension in step (i) is at a temperature of: about 20° C. to about 90° C., about 30° C. to about 80° C., about 40° C. to about 75° C., about 45° C. to about 60° C., or about 50° C. According to any aspect or embodiment of the process, the diisopropyl ether may be used in an amount of: about 20 ml to about 80 ml, about 30 ml to about 70 ml, or about 50 ml, per gram of the Denifanstat. In any embodiment of this process, step (ii) is carried out at a temperature of: about 20° C. to about 90° C., about 30° C. to about 80° C., about 40° C. to about 75° C., or about 50° C. In any embodiment of the process, step (ii) comprises stirring the suspension for period of: about 15 hour to 72 hours, or about 24 hours to about 60 hours, or about 48 hours. The Form DF1 Form of Denifanstat may be isolated in step (iii) by any suitable method, such as by filtration, centrifuge or decantation, preferably by filtration. The solid may be dried, optionally under reduced pressure. In any embodiment of this process, the crystalline DF1 Form of Denifanstat may be dried under vacuum. The crystalline DF1 Form of Denifanstat may be dried under vacuum, typically at a temperature of: about 40° C. to about 80° C., about 50° C. to about 70° C., or at room temperature, preferably at about 25° C. The drying may be carried out for any suitable time to remove the solvent, typically: about 1 to about 5 hours, about 1.5 hours to about 4 hours, or about 30 minutes.

Alternatively, Form DF1 of Denifanstat according to any aspect or embodiment may be prepared by a process comprising:

Alternatively, Form DF1 of Denifanstat according to any aspect or embodiment may be prepared by crystallising Denifanstat from a solution in a C₁ to C₄ alcohol (preferably a C₁ to C₃ alcohol, particularly ethanol or isopropanol, and more particularly, isopropanol. The crystallisation may comprise combining the solution with an antisolvent (preferably wherein the antisolvent is a C₅ to C₈ alkane, more preferably a C₆ to C₇ alkane, and most preferably n-heptane). Form DF1 of Denifanstat according to any embodiment of the disclosure may be prepared by a process comprising:
a) providing a solution of Denifanstat in isopropyl alcohol;
b) combining the solution with n-heptane;
c) optionally isolating the Form DF1 of Denifanstat; and
d) optionally drying.

In any embodiment of this process, the solution in step (a) may be prepared by dissolving Denifanstat in isopropyl alcohol at temperature, preferably: about 18° C. to about 65° C., about 20° C. to about 50° C., about 20° C. to about 30° C., or about 25° C. According to any aspect or embodiment of the process, the isopropyl alcohol may be used in an amount of: about 6 ml to about 30 ml, about 8 ml to about 20 ml, about 9 ml to about 15 ml, or about 10 ml to about 12 ml, per gram of Denifanstat. According to any aspect or embodiment of the process, the n-heptane may be added a temperature of about 18° C. to about 45° C., about 20° C. to about 40° C., about 20° C. to about 30° C., or about 25° C. According to any aspect or embodiment of the process, the n-heptane may be preferably used in an in an amount of about 10 ml to about 80 ml, about 20 ml to about 70 ml, about 25 ml to about 60 ml, about 30 ml to about 55 ml, or about 33 ml to about 35 ml, per gram of Denifanstat. According to any aspect or embodiment of the process, the ratio (vol:vol) of isopropyl alcohol to n-heptane is: about 5:1 to about 1:5, about 4:1 to about 1:4, about 1:2 to about 1:4, or about 1:3 to about 1:3.5. Step (b) may comprise adding the n-heptane to the solution of Denifanstat in isopropyl alcohol, or adding the solution of Denifanstat in isopropyl alcohol to the n-heptane. Preferably step (b) comprises adding the Denifanstat in isopropyl alcohol to the n-heptane. The addition of the solution of Denifanstat in isopropyl alcohol may be carried out in one portion, or portionwise, or dropwise. In any embodiment of the process, following the combining of the solution of the Denifanstat in isopropyl alcohol and the n-heptane, the mixture may be maintained for a sufficient time to prepare Denifanstat Form DF1. The mixture may be seeded, preferably with Denifanstat Form DF1, and optionally in an amount of about 1.0 wt % to about 8.0 wt %, about 2.0 wt % to about 6.0 wt %, or about 5 wt %. According to any aspect or embodiment of the process, the Denifanstat Form DF1 seed may be added in an amount of about 0.10 to about 0.03 g, or about 0.05 g, per gram of Denifanstat starting material. In any embodiment of the process, the mixture may be maintained preferably at a temperature of: about 18° C. to about 45° C., about 20° C. to about 40° C., about 20° C. to about 30° C., or about 25° C., and preferably for a period of: about 1 to about 12 hours, about 1 to about 10 hours, about 1.5 to about 8 hours, or about 2 to about 6 hours. The may be added to it. The Denifanstat Form DF1 may be isolated, preferably by any suitable process, such as by decantation, filtration or by centrifuge, preferably by filtration. In any embodiment of this process, the crystalline Form DF1 of Denifanstat may be dried under suction. The crystalline Form DF1 of Denifanstat may be dried, typically at a temperature of about 20° C. to about 60° C., about 25° C. to about 55° C., about 25° C. The drying may be carried out for any suitable time to remove the solvent, typically about 1 to about 5 hours, about 1.5 hours to about 4 hours, or about 2 hours to about 3 hours, or about 30 minutes.

In any aspect or embodiment, the processes as described herein for producing Form DF2 of Denifanstat may further comprise combining the crystalline form with at least one excipient to prepare a pharmaceutical composition.

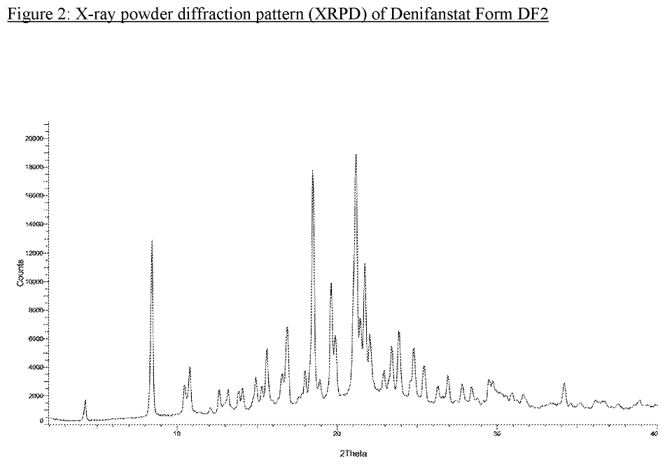

The present disclosure includes a crystalline polymorph of Denifanstat, designated DF2. The crystalline Form DF2 of Denifanstat may be characterized by data selected from one or more of the following: an X-ray powder diffraction pattern substantially as depicted in FIG. 2; an X-ray powder diffraction pattern having peaks at 19.8, 23.4 and 25.4 degrees 2-theta±0.2 degrees 2-theta; and combinations of these data.

Crystalline Form DF2 of Denifanstat may be further characterized by an X-ray powder diffraction pattern having peaks at 19.8, 23.4 and 25.4 degrees 2-theta±0.2 degrees 2-theta, and also having any one, two, three, four or five additional peaks selected from 4.3, 8.4, 21.2, 22.9 and 23.9 degrees 2-theta±0.2 degrees 2-theta.

Alternatively, Crystalline Form DF2 of Denifanstat may be characterized by an X-ray powder diffraction pattern having peaks at 4.3, 8.4, 21.2, 22.9 and 23.9 degrees 2-theta±0.2 degrees 2-theta. Crystalline Form DF2 of Denifanstat may be further characterized by an X-ray powder diffraction pattern having peaks at 4.3, 8.4, 21.2, 22.9 and 23.9 degrees 2-theta±0.2 degrees 2-theta, and also having any one, two, three, or four additional peaks selected from 10.8, 16.9, 19.6 and 25.4 degrees 2-theta±0.2 degrees 2-theta.

Crystalline Form DF2 of Denifanstat may be characterized by an X-ray powder diffraction pattern having peaks at 4.3, 8.4, 10.8, 16.9, 19.6, 21.2, 22.9, 23.9 and 25.4 degrees 2-theta±0.2 degrees 2-theta.

In any aspect or embodiment of the present disclosure, crystalline Form DF2 of Denifanstat may be characterized by an X-ray powder diffraction pattern as described in any of the embodiments described herein, and wherein the X-ray powder diffraction pattern also has an absence of peaks at: 0 to 3.8 degrees 2-theta±0.2 degrees 2-theta, or 2.0 to 3.8 degrees 2-theta±0.2 degrees 2-theta, or 2.0 to 3.5 degrees±0.2 degrees 2-theta. Alternatively or additionally, according to any aspect or embodiment of the present disclosure, crystalline Form DF2 of Denifanstat may be characterized by an X-ray powder diffraction pattern as described in any of the embodiments described herein, and wherein the X-ray powder diffraction pattern also has an absence of peaks at: 4.8 to 7.9 degrees 2-theta±0.2 degrees 2-theta, or 5.0 to 7.5 degrees 2-theta±0.2 degrees 2-theta.

In any aspect of aspect or embodiment of the present disclosure, crystalline DF2 of Denifanstat may be characterized by:
an X-ray powder diffraction pattern having peaks at: 19.8, 23.4 and 25.4 degrees 2-theta±0.2 degrees 2-theta; or
an X-ray powder diffraction pattern having peaks at: 19.8, 23.4 and 25.4 degrees 2-theta±0.2 degrees 2-theta, and also having any one, two, three, four or five additional peaks selected from: 4.3, 8.4, 21.2, 22.9 and 23.9 degrees 2-theta±0.2 degrees 2-theta or
an X-ray powder diffraction pattern having peaks at: 4.3, 8.4, 21.2, 22.9 and 23.9 degrees 2-theta±0.2 degrees 2-theta; or
an X-ray powder diffraction pattern having peaks at: 4.3, 8.4, 21.2, 22.9 and 23.9 degrees 2-theta±0.2 degrees 2-theta, and also having any one, two, three, or four additional peaks selected from: 10.8, 16.9, 19.6 and 25.4 degrees 2-theta±0.2 degrees 2-theta; or
an X-ray powder diffraction pattern having peaks at: 4.3, 8.4, 10.8, 16.9, 19.6, 21.2, 22.9, 23.9 and 25.4 degrees 2-theta±0.2 degrees 2-theta;
   wherein in each case, the X-ray powder diffraction pattern has an absence of peaks at: 0 to 3.8 degrees 2-theta±0.2 degrees 2-theta (particularly 2.0 to 3.5 degrees±0.2 degrees 2-theta); and/or an absence of peaks at: 4.8 to 7.9 degrees 2-theta±0.2 degrees 2-theta (particularly 5.0 to 7.5 degrees 2-theta±0.2 degrees 2-theta).

In one embodiment of the present disclosure, crystalline Form DF2 of Denifanstat is isolated.

Crystalline Form DF2 of Denifanstat may be anhydrous form.

Crystalline Form DF2 of Denifanstat may be characterized by each of the above characteristics alone/or by all possible combinations, e.g., an XRPD pattern having peaks at 19.8, 23.4 and 25.4 degrees 2-theta±0.2 degrees 2-theta; an XRPD pattern having peaks at 4.3, 8.4, 21.2, 22.9 and 23.9 degrees 2-theta±0.2 degrees 2-theta; an XRPD pattern as depicted in FIG. 2, and combinations thereof.

Crystalline Form DF2 of Denifanstat according to any aspect or embodiment of the disclosure may be polymorphically pure or as substantially free of any other solid state (or polymorphic) forms of Denifanstat.

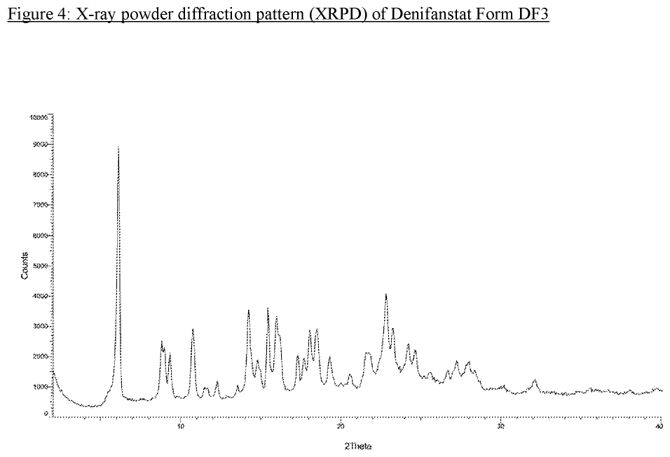

The present disclosure includes a crystalline polymorph of Denifanstat, designated DF3. The crystalline Form DF3 of Denifanstat may be characterized by data selected from one or more of the following: an X-ray powder diffraction pattern substantially as depicted in FIG. 4; an X-ray powder diffraction pattern having peaks at 19.8, 23.4 and 25.4 degrees 2-theta±0.2 degrees 2-theta; and combinations of these data.

Crystalline Form DF3 of Denifanstat may be further characterized by an X-ray powder diffraction pattern having peaks at 19.8, 23.4 and 25.4 degrees 2-theta±0.2 degrees 2-theta, and also having any one, two, three, four or five additional peaks selected from 6.1, 8.8, 9.3, 16.0, 19.3 and 24.2 degrees 2-theta±0.2 degrees 2-theta.

Alternatively, Crystalline Form DF3 of Denifanstat may be characterized by an X-ray powder diffraction pattern having peaks at 6.1, 8.8, 9.3, 16.0, 19.3 and 24.2 degrees 2-theta±0.2 degrees 2-theta. Crystalline Form DF3 of Denifanstat may be further characterized by an X-ray powder diffraction pattern having peaks at 6.1, 8.8, 9.3, 16.0, 19.3 and 24.2 degrees 2-theta±0.2 degrees 2-theta, and also having any one, two, three, or four additional peaks selected from 14.2, 15.5, 22.8 and 24.6 degrees 2-theta #0.2 degrees 2-theta.

Crystalline Form DF3 of Denifanstat may be characterized by an X-ray powder diffraction pattern having peaks at 6.1, 8.8, 9.3, 14.2, 15.5, 16.0, 19.3, 22.8, 24.2 and 24.6 degrees 2-theta±0.2 degrees 2-theta.

In any aspect or embodiment of the present disclosure, crystalline Form DF3 of Denifanstat may be characterized by an X-ray powder diffraction pattern as described in any of the embodiments described herein, and wherein the X-ray powder diffraction pattern also has an absence of peaks at: 0 to 5.0 degrees 2-theta±0.2 degrees 2-theta, or 2.0 to 5.0 degrees 2-theta±0.2 degrees 2-theta, or 3.0 to 4.0 degrees±0.2 degrees 2-theta. Alternatively or additionally, according to any aspect or embodiment of the present disclosure, crystalline Form DF3 of Denifanstat may be characterized by an X-ray powder diffraction pattern as described in any of the embodiments described herein, and wherein the X-ray powder diffraction pattern also has an absence of peaks at: 6.5 to 8.0 degrees 2-theta±0.2 degrees 2-theta, or 6.6 to 7.2 degrees 2-theta±0.2 degrees 2-theta.

In any aspect of aspect or embodiment of the present disclosure, crystalline DF3 of Denifanstat may be characterized by:
an X-ray powder diffraction pattern having peaks at: 19.8, 23.4 and 25.4 degrees 2-theta±0.2 degrees 2-theta; and combinations of these data; or
an X-ray powder diffraction pattern having peaks at: 19.8, 23.4 and 25.4 degrees 2-theta±0.2 degrees 2-theta, and also having any one, two, three, four or five additional peaks selected from: 6.1, 8.8, 9.3, 16.0, 19.3 and 24.2 degrees 2-theta±0.2 degrees 2-theta; or
an X-ray powder diffraction pattern having peaks at: 6.1, 8.8, 9.3, 16.0, 19.3 and 24.2 degrees 2-theta±0.2 degrees 2-theta; or
an X-ray powder diffraction pattern having peaks at: 6.1, 8.8, 9.3, 16.0, 19.3 and 24.2 degrees 2-theta±0.2 degrees 2-theta, and also having any one, two, three, or four additional peaks selected from: 14.2, 15.5, 22.8 and 24.6 degrees 2-theta±0.2 degrees 2-theta; or
an X-ray powder diffraction pattern having peaks at: 6.1, 8.8, 9.3, 14.2, 15.5, 16.0, 19.3, 22.8, 24.2 and 24.6 degrees 2-theta±0.2 degrees 2-theta.
   wherein in each case, the X-ray powder diffraction pattern also has an absence of peaks at: 0 to 5.0 degrees 2-theta±0.2 degrees 2-theta (particularly 3.0 to 4.0 degrees±0.2 degrees 2-theta); and/or an absence of peaks at: 6.5 to 8.0 degrees 2-theta±0.2 degrees 2-theta (particularly 6.6 to 7.2 degrees 2-theta±0.2 degrees 2-theta).

In one embodiment of the present disclosure, crystalline Form DF3 of Denifanstat is isolated.

Crystalline Form DF3 of Denifanstat may be characterized by each of the above characteristics alone/or by all possible combinations, e.g., an XRPD pattern having peaks at 19.8, 23.4 and 25.4 degrees 2-theta±0.2 degrees 2-theta; an XRPD pattern having peaks at 6.1, 8.8, 9.3, 16.0, 19.3 and 24.2 degrees 2-theta±0.2 degrees 2-theta; an XRPD pattern as depicted in FIG. 4, and combinations thereof.

Crystalline Form DF3 of Denifanstat according to any aspect or embodiment of the disclosure may be polymorphically pure or as substantially free of any other solid state (or polymorphic) forms of Denifanstat.

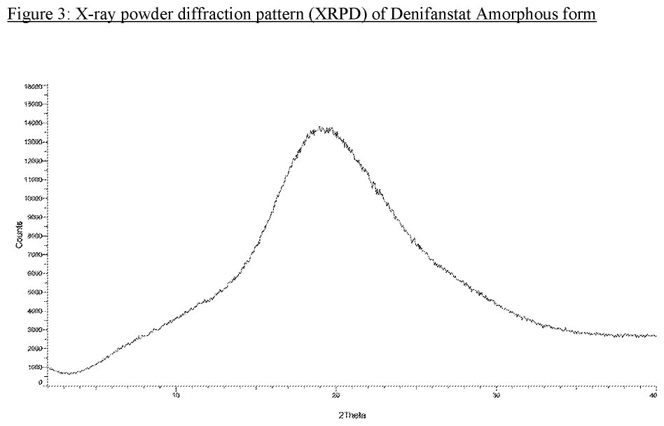

The present disclosure includes amorphous form of Denifanstat. The amorphous Form of Denifanstat may be characterized by an X-ray powder diffraction pattern substantially as depicted in FIG. 3.

The amorphous Form of Denifanstat may be isolated as pure amorphous form. The amorphous form of Denifanstat is specifically pure from crystalline forms, and the meaning is as defined above. Accordingly, amorphous form of Denifanstat according to any aspect or embodiment of the disclosure may be polymorphically pure and substantially free of any crystalline forms of Denifanstat.

The present disclosure further comprises a process for preparation of amorphous form of Denifanstat. According to any aspect or embodiment, amorphous form of Denifanstat may be prepared by a process comprising precipitation of amorphous Denifanstat from mixture comprising an alcohol (preferably a C₁ to C₄ alcohol, or a C₁-C₃ alcohol, particularly methanol) and water. According to any aspect or embodiment, amorphous form of Denifanstat may be prepared by a process comprising precipitation of amorphous Denifanstat from mixture comprising methanol as a solvent and water as an anti-solvent. The precipitation may be carried out rapidly, for example, by the fast addition of the anti-solvent, by the addition of cold anti-solvent (for example wherein the anti-solvent is at a temperature of: about 0° C. to about 10° C., about 0° C. to about 5° C., about 0° C. to about 2° C., or about 0° C.), or by the addition of the methanol solution to the anti-solvent, particularly wherein the antisolvent is cooled (for example wherein the anti-solvent is cooled to a temperature of: about 0° C. to about 10° C., about 0° C. to about 5° C., about 0° C. to about 2° C., or about 0° C.). The precipitation comprises providing a solution of the Denifanstat in the alcohol (preferably methanol) and combining the solution with water to obtain a precipitate. The solution of Denifanstat in the alcohol (preferably methanol), may be at a temperature of: about 20° C. to about 90° C., about 30° C. to about 80° C., about 40° C. to about 75° C., about 50° C. to about 65° C. or about 60° C. In any embodiment, the water may be added to a solution of Denifanstat in methanol, preferably wherein the water is at a temperature of: about 0° C. to about 5° C., about 0° C. to about 2° C., or about 0° C. In any embodiment, the mixture may maintained preferably at a temperature of: about −5° C. to about 15° C., about −2° C. to about 10° C., about 0° C. to about 5° C., or about 0° C., and preferably for a period of: about 1 to about 12 hours, about 1 to about 8 hours, about 1.5 to about 6 hours, about 2 to about 4 hours, or about 3 hours.

In any aspect or embodiment, the processes as described herein for producing the Denifanstat forms as described in any aspect or embodiment, may further comprise combining the crystalline form with at least one excipient to prepare a pharmaceutical composition.

The process for preparing amorphous form of Denifanstat according to any embodiment may further comprise recovering the said amorphous form. The recovery may be done, for example, by filtering the precipitate, for example by vacuum filtration; and drying. Preferably, drying is done under reduced pressure, particularly using vacuum tray drier, typically at a temperature of from about 60° C., for example for a period of about 16 hours. Particularly, the drying may be carried out for any suitable time to remove the solvent, typically: about 1 to about 48 hours, about 8 hours to about 30 hours, about 10 hours to about 20 hours, or about 16 hours.

The above crystalline polymorphs can be used to prepare other crystalline polymorphs of Denifanstat, Denifanstat salts and their solid state forms.

The present disclosure encompasses a process for preparing other solid state forms of Denifanstat, Denifanstat salts and their solid state forms thereof.

The present disclosure provides the above described crystalline polymorphs of Denifanstat for use in the preparation of pharmaceutical compositions comprising Denifanstat and/or crystalline polymorphs thereof.

The present disclosure also encompasses the use of crystalline polymorphs of Denifanstat of the present disclosure for the preparation of pharmaceutical compositions of crystalline polymorph Denifanstat and/or crystalline polymorphs thereof.

The present disclosure includes processes for preparing the above mentioned pharmaceutical compositions. The processes includes combining any one or a combination of the crystalline polymorphs of Denifanstat of the present disclosure with at least one pharmaceutically acceptable excipient.

Pharmaceutical combinations or formulations of the present disclosure contain any one or a combination of the solid state forms of Denifanstat of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Denifanstat and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Denifanstat can be administered. Denifanstat may be formulated for administration to a mammal, in embodiments to a human, by injection. Denifanstat can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Denifanstat and the pharmaceutical compositions and/or formulations of Denifanstat of the present disclosure can be used as medicaments, in embodiments in the treatment of Glioblastoma, Acne; Astrocytoma; Breast cancer, Non-alcoholic steatohepatitis, Non-small cell lung cancer, Liver disorders, Colorectal cancer, and Solid tumours.

The present disclosure also provides methods of treating Glioblastoma, Acne; Astrocytoma; Breast cancer, Non-alcoholic steatohepatitis, Non-small cell lung cancer, Liver disorders, Colorectal cancer, and Solid tumours by administering a therapeutically effective amount of any one or a combination of the crystalline polymorphs of Denifanstat of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

Solid State ¹³C-NMR (¹³C CP/MAS NMR) Method

Solid-state NMR spectra were measured at 11.7 T using a Bruker Avance III HD 500 US/WB NMR spectrometer (Karlsruhe, Germany, 2013) with 3.2 mm probehead. The 13C CP/MAS NMR spectra employing cross-polarization were acquired using the standard pulse scheme at spinning frequency of 15 kHz and a room temperature (300 K). The recycle delay was 8 s and the cross-polarization contact time was 2 ms. The ¹³C scale was referenced to α-glycine (176.03 ppm for ¹³C). Frictional heating of the spinning samples was offset by active cooling, and the temperature calibration was performed with Pb(NO3)2. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δυ½ was less than 3.5 Hz at 250 ms of acquisition time).

### Powder X-Ray Diffraction (“XRPD”) Method

X-ray diffraction was performed on X-Ray powder diffractometer:
Bruker D8 Advance; CuKα radiation (λ=1.5418 Å); Lynx eye detector; laboratory temperature 22-25° C.; PMMA specimen holder ring with silicon low background. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring with silicon low background.
All X-Ray Powder Diffraction peak values are calibrated with regard to standard silicon spiking in the sample.

### HPLC Method

Column: Xbridge C₁₈ (4.6 mm×150 mm×5 μm)
Mobile phase:A: H2O (0.05% TFA); B: Acetonitrile
Gradient:

| | Flow rate | | |
|---|---|---|---|
| Time (min) | (mL/min) | A % | B % |
| 0.01 | 1.0 | 95 | 5 |
| 4 | 1.0 | 5 | 95 |
| 8 | 1.0 | 5 | 95 |
| 10 | 1.0 | 95 | 5 |
| 15 | 1.0 | 95 | 5 |

Wavelength: UV at 220 nm
Column Oven Temperature: 40° C.
Injected volume: 1 μL
Sample preparation: Samples were dissolved in acetonitrile with concentration 0.5 mg/ml the retention time of Denifanstat peak is about 4.3 min

### EXAMPLES

### Preparation of Starting Materials

Denifanstat can be prepared according to methods known from the literature, for example U.S. Pat. No. 8,871,790.

### Example 1: Preparation of Denifanstat Form DF1

Denifanstat amorphous form (0.05 g) was taken in a 10 mL vial and was suspended in 2.5 mL of Diisopropyl ether at temperature of about 50° C. for period of about 2 days. Solid obtained was filtered and suction dried at temperature of about 25° C. for period of about 30 minutes. The obtained solid was analyzed by XRPD. Crystalline Denifanstat Form DF1 was obtained. An XRPD pattern is shown in FIG. 1.

### Example 2: Preparation of Denifanstat Form DF2

Denifanstat amorphous form (0.05 g) was taken in a 1 mL vial and was dissolved in 0.2 mL of isoamyl alcohol at temperature of about 60° C. The clear solution was added to 0.6 mL of n-decane which was kept at temperature of about 0° C. and stirred at temperature of about 0° C. for period of about 2 hours. The clear solution was maintained at temperature of about 25° C. with stirring for period of about 7 days. Solid obtained was filtered and suction dried at temperature of about 25° C. for period of about 30 minutes. The obtained solid was analyzed by XRPD. Crystalline Denifanstat Form DF2 was obtained. An XRPD pattern is shown in FIG. 2.

### Example 3: Preparation of Denifanstat Amorphous Form

Denifanstat (5.0 g) was taken in a 100 mL round bottom flask and was dissolved in 10 mL of methanol at temperature of about 60° C. The clear solution was filtered and added to 40 mL of water which was kept at temperature of about 0° C. and stirred for period of about 3 hours at temperature of about 0° C. Solid obtained was filtered and suction dried at temperature of about 25° C. for period of about 30 minutes and was further dried under vacuum tray drier at temperature of about 60° C. for period of about 16 hours. The obtained solid was analyzed by XRPD. Denifanstat Amorphous Form was obtained. An XRPD pattern is shown in FIG. 3. (HPLC Purity: 99.38%)

### Example 4: Preparation of Denifanstat Form DF3

Denifanstat (0.02 g) was taken in a 1 mL vial and was dissolved in 0.12 mL of Ethanol at temperature of about 65° C. The clear solution was added to 0.5 mL of p-xylene which was kept at temperature of about 0° C. to about 5° C. and stirred for period of about 2 hours at temperature of about 0° C. to about 5° C. The clear solution was maintained at temperature of about 25° C. with stirring for period of about 2 days. Solid obtained was filtered and suction dried at temperature of about 25° C. for period of about 30 minutes. The obtained solid was analyzed by XRPD. Crystalline Denifanstat Form DF3 was obtained. An XRPD pattern is shown in FIG. 4.

### Example 5: Preparation of Denifanstat Form DF1

Denifanstat (0.03 g) was taken in a 1 mL vial and was dissolved in 0.35 mL of Isopropyl alcohol at temperature of about 25° C. The clear solution was added to 1.0 mL of n-heptane which was kept at temperature of about 25° C. and stirred at temperature of about 25° C. for period of about 4 to about 6 hours. Solid obtained was filtered and suction dried at temperature of about 25° C. for period of about 30 minutes. The obtained solid was further dried under vacuum at 60° C. for about 60 minutes. The obtained solid was analyzed by XRPD. Crystalline Denifanstat Form DF1 was obtained.

### Example 6: Preparation of Denifanstat Form DF1

Denifanstat (0.5 g) was taken in a 10 mL vial and was dissolved in 5 mL of Isopropyl alcohol at temperature of about 25° C. The clear solution was added drop by drop to 17 mL of n-heptane which was kept at temperature of about 25° C. along with 5% DF1 seed (0.025 g, 5 wt % based on Denifanstat starting material) and stirred at temperature of about 25° C. for period of about 2 to about 4 hours. Solid obtained was filtered and dried suction at 25° C. for 30 minutes. The obtained solid was further dried under vacuum at 60° C. for about 60 minutes. The obtained solid was analyzed by XRPD. Crystalline Denifanstat Form DF1 was obtained.

### Example 7: Preparation of Denifanstat Form DF1

Denifanstat amorphous form (0.05 g) was taken in a 10 mL vial and was suspended in 2.5 mL of diisopropyl ether at temperature of about 50° C. for period of about 2 days. The solid obtained was filtered and suction dried at temperature of about 25° C. for period of about 30 minutes. The obtained solid was further dried under vacuum at 60° C. for about 60 minutes and was analyzed by XRPD. Crystalline Denifanstat Form DF1 was obtained.

### Example 8: Stability of Denifanstat Form DF1

### Storage Stability at Different Relative Humidities

Samples Denifanstat Form DF1 was subjected to conditions of different relative humidities at different temperatures. XRPD analysis was performed on the samples after 6 months. The results are shown in Table 1 below:

TABLE 1

Conditions (6 months)

| Denifanstat | 25 °C, | 40 °C, |
|---|---|---|
| Form DF1 | 60% RH | 75% RH |
| XRPD analysis results | No change | No change |

The results demonstrate that Form DF1 of Denifanstat is stable after exposure to high and low relative humidity at different temperatures for at least 6 months, indicating that this crystalline form has good storage stability.

### Grinding Experiments

Samples of Form DF1 of Denifanstat were subjected to strong grinding, and to solvent drop grinding in water. Grinding was carried out on the sample alone, or in the presence of water. In these experiments, about 20 mg of the sample is placed in a mortar and ground with a pestle for 2 minutes. The water, when used, as added to the crystalline material before grinding, in a volume of about 10 microlitres. XRPD analysis performed on the samples after the grinding experiment, confirmed no change in the starting material (Table 2):

TABLE 2
Form DFI of Denifanstat

| Condition | XRPD analysis results |
|---|---|
| Strong grinding | No change |
| Solvent-drop grinding | No change |
| (water) | |

The results demonstrate that Form DF1 of Denifanstat is resistant to polymorphic changes and is highly suitable for preparing pharmaceutical formulations.

### Thermal Stability

A sample of Form DF1 of Denifanstat was subjected to heating up to 100° C. for 30 minutes. XRPD analysis of the sample confirmed no change in the starting material (Table 3):

TABLE 3
Form DF1 of Denifanstat

| Condition | Result |
|---|---|
| Heating 100 °C, | Stable |
| 30 minutes | |

### Stability to Compression

A sample of Form DF1 of Denifanstat was subjected to a pressure of 2 tons (Atlas® Autopress hydraulic press, set to 2 tons). XRPD analysis was performed on the samples after 1-5 minutes. The results are shown in Table 4 below:

TABLE 4

| XRPD analysis | Pressure |
|---|---|
| results | (2 tons) |
| Form DF1 of | No change |
| Denifanstat | |

Accordingly, Form DF1 of Denifanstat is stable under high pressure conditions, making it highly suitable for pharmaceutical processing.

1. A crystalline Denifanstat.

2. A crystalline Denifanstat which is characterized by an X-ray powder diffraction pattern having peaks at 19.8, 23.4 and 25.4 degrees 2-theta±0.2 degrees 2-theta.

3. The crystalline Denifanstat according to claim 1, which is characterized by data selected from one or more of the following:
a) an XRPD pattern having peaks at 19.8, 23.4 and 25.4 degrees 2-theta±0.2 degrees 2-theta;
b) an XRPD pattern as depicted in FIG. 1;
c) a solid state ¹³C NMR spectrum having peaks at 13.6, 25.7, 35.1, 39.3, 47.9, 109.3, 135.0, 147.0, 150.4, 154.4, 162.7 and 172.0 ppm±0.2 ppm;
d) a solid state ¹³C NMR spectrum having the following chemical shift absolute differences from a reference peak at 18.5 ppm±2 ppm of 4.9, 7.2, 16.6, 20.8, 29.4, 90.8, 116.5, 128.5, 131.9, 135.9, 144.2 and 153.5 ppm±0.1 ppm;
e) a solid-state ¹³C NMR spectrum substantially as depicted in FIG. 5a, 5b or 5c; and
f) combinations of two or more of: a, b, c, d, and e.

4. The crystalline Denifanstat according to claim 1, which is characterized by an X-ray powder diffraction pattern having peaks at 19.8, 23.4 and 25.4 degrees 2-theta±0.2 degrees 2-theta, and also having any one, two, three, four or five additional peaks selected from 6.3, 14.5, 18.3, 20.4 and 20.9 degrees 2-theta±0.2 degrees 2-theta.

5. The crystalline Denifanstat according to claim 1, which is characterized by an X-ray powder diffraction pattern having peaks at 6.3, 14.5, 18.3, 20.4 and 20.9 degrees 2-theta±0.2 degrees 2-theta.

6. The crystalline Denifanstat according to claim 1, characterized by an X-ray powder diffraction pattern having peaks at 6.3, 14.5, 18.3, 20.4 and 20.9 degrees 2-theta±0.2 degrees 2-theta and also having any one, two, three, or four additional peaks selected from 12.7, 17.7, 19.8 and 22.4 degrees 2-theta±0.2 degrees 2-theta.

7. The crystalline Denifanstat according to claim 1, characterized by an X-ray powder diffraction pattern having peaks at 6.3, 12.7, 14.5, 17.7, 18.3, 19.8, 20.4, 20.9, 22.4, 23.4 and 25.4 degrees 2-theta±0.2 degrees 2-theta.

8. The crystalline Denifanstat according to claim 1, wherein the crystalline form is an anhydrous form.

9. The crystalline Denifanstat according to claim 1, which contains: no more than about 20%, no more than about 10%, no more than about 5%, no more than about 2%, no more than about 1%, or about 0% of amorphous Denifanstat.

10. A pharmaceutical formulation comprising the crystalline of Denifanstat according to claim 1.

11. (canceled)

12. The pharmaceutical formulation according to claim 10, further comprising at least one pharmaceutically acceptable excipient.

13. A process for preparing a pharmaceutical formulation, the process comprising: combining the crystalline Denifanstat according to claim 1 with at least one pharmaceutically acceptable excipient.

14. (canceled)

15. (canceled)

16. A method of treating, comprising administering a therapeutically effective amount of the crystalline of Denifanstat according to claim 1 to a subject in need of the treatment of a condition.

17. The method of treating according to claim 16, wherein the condition is at least one of Glioblastoma, Acne; Astrocytoma, Breast cancer; Non-alcoholic steatohepatitis, Non-small cell lung cancer, Liver disorders, Colorectal cancer, or Solid tumours.

18. (canceled)
